(19) United States
(12) Patent Application Publication
(10) Pub. No.: US 2026/0217705 A1
(43) Pub. Date: Jul. 30, 2026

(54) POLYMORPHS OF THE HYDROBROMIDE SALT OF LINAPRAZAN GLURATE

(71) Applicant: CINCLUS PHARMA HOLDING AB (PUBL), Stockholm (SE)

(72) Inventors: Mikael Hillgren, Uppsala (SE); Lars-Erik Briggner, Hoor (SE); Emelie Haettner, Lund (SE)

(21) Appl. No.: 19/147,011

(22) PCT Filed: Jan. 11, 2024
(86) PCT No.: PCT/EP2024/050572

§ 371 Date: Jul. 10, 2025

(30) Foreign Application Priority Data
Jan. 11, 2023 (SE) 2330016-3

(51) Int. Cl. C07D 471/04; A61K 31/437

(52) U.S. Cl. CPC C07D 471/04; A61K 31/437

(57) ABSTRACT

The present invention relates to polymorphs of the hydrobromide salt of 5-{2-[({8-[(2,6-dimethylbenzyl)amino]-2,3-dimethylimidazo[1,2-a]pyridine-6-yl}carbonyl)-amino]ethoxy}-5-oxopentanoic acid (linaprazanglurate), more specifically Form A, Form B and Form C of the HBr salt of linaprazan glurate. The invention also relates to pharmaceutical compositions comprising such polymorphs, and to the use of these polymorphs in the treatment or prevention of gastrointestinal inflammatory diseases or gastric acid related diseases, in particular erosive gastroesophageal reflux disease (eGERD).

### TECHNICAL FIELD

The present invention relates to polymorphs of the hydrobromide salt of 5-{2-[({8-[(2,6-dimethylbenzyl)amino]-2,3-dimethylimidazo[1,2-a]pyridine-6-yl}carbonyl)-amino]ethoxy}-5-oxopentanoic acid (linaprazan glurate), more specifically Form A, Form B and Form C of the HBr salt of linaprazan glurate. The invention also relates to pharmaceutical compositions comprising such polymorphs, and to the use of these polymorphs in the treatment or prevention of gastrointestinal inflammatory diseases or gastric acid related diseases, in particular erosive gastroesophageal reflux disease (eGERD).

### BACKGROUND

The compound linaprazan glurate (5-{2-[({8-[(2,6-dimethylbenzyl)amino]-2,3-dimethylimidazo[1,2-a]pyridine-6-yl}carbonyl)-amino]ethoxy}-5-oxopentanoic acid; previously known as X842) is disclosed in WO 2010/063876. Its structure is shown below. It is a potassium-competitive acid blocker (P-CAB), which competitively inhibits the gastric hydrogen potassium pump (H⁺/K⁺ ATPase) in the parietal cells. Linaprazan glurate may therefore be used to control the secretion of gastric acid in the stomach.

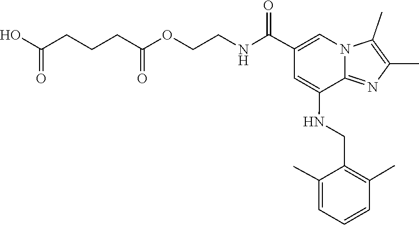 [SMILES: Cc1cccc(C)c1CNc1cc(C(=O)NCCOC(=O)CCCC(=O)O)cn2c(C)c(C)nc12]

Linaprazan glurate is a prodrug of linaprazan, which was disclosed in WO 99/55706 and previously studied in Phase I and II studies. These studies showed that linaprazan was well tolerated, with a fast onset of action and full effect at first dose. However, linaprazan was quickly eliminated from the body and had too short duration of acid inhibition. In comparison, linaprazan glurate has a longer half-life in the body and shows total control of the gastric acid production for a longer time compared to linaprazan. A clinical Phase I study has shown that administration of a single dose of linaprazan glurate can maintain the intragastric acidity above pH 4 for 24 hours. Linaprazan glurate is therefore tailored for patients with severe erosive gastroesophageal reflux disease (eGERD).

For use in pharmaceutical preparations, it is desirable that the active pharmaceutical ingredient (API) is in a highly crystalline form. Non-crystalline (i.e., amorphous) materials may contain higher levels of residual solvents, which is undesirable. Also, because of their lower chemical and physical stability, as compared with crystalline material, amorphous materials may display faster decomposition and may spontaneously form crystals with a variable degree of crystallinity. This may result in unreproducible solubility rates and difficulties in storing and handling the material.

Two crystalline forms of the free base of linaprazan glurate are disclosed in CN 10627915. Forms A and B of the free base were found to be anhydrates, and Form A was shown to have a very low hygroscopicity. While Form A has good physical and chemical stability and can be obtained with high crystallinity, it is practically insoluble in water at pH 6.8, and only slightly soluble at pH 1. The low solubility restricts the development of formulations having desirable properties.

There is therefore a need for further crystalline forms of linaprazan glurate that have better properties than amorphous linaprazan glurate and the previously disclosed crystalline forms thereof. In particular, it is an object of the present invention to provide a stable crystalline form of linaprazan glurate that has good solubility, contains low levels of residual solvents, has a high chemical stability and low hygroscopicity and can be obtained in high levels of crystallinity.

### BRIEF DESCRIPTION OF THE DRAWINGS

FIG. 1 shows the X-ray powder diffractogram of Form A of the HBr salt of linaprazan glurate, as obtained from the synthesis described in Example 1.

FIG. 2 shows the X-ray powder diffractogram of Form B of the HBr salt of linaprazan glurate, as obtained from a slurry in methanol/toluene 4:1 (“sample 1”).

FIG. 3 shows the X-ray powder diffractogram of Form B of the HBr salt of linaprazan glurate, as obtained from a slurry in methanol/water 4:1, showing additional peaks of a presumed channel solvate (“sample 2”).

FIG. 4 shows the X-ray powder diffractogram of Form C of the HBr salt of linaprazan glurate, as obtained from a slurry in DMF/toluene 1:1.

FIG. 5 shows the thermogravimetric analysis (TGA) weight loss curve of Form A (as obtained in Example 1).

FIG. 6 shows the TGA weight loss curve of Form B (sample 1).

FIG. 7 shows the TGA weight loss curve of Form C.

FIG. 8 shows the differential scanning calorimetry (DSC) thermogram of Form A.

FIG. 9 shows the differential scanning calorimetry (DSC) thermogram of Form B.

FIG. 10 shows the differential scanning calorimetry (DSC) thermogram of Form C.

FIG. 11 shows the dynamic vapour sorption (DVS) weight change plot (A) and the DVS isotherm plot (B) for Form A.

FIG. 12 shows the DVS weight change plot (A) and the DVS isotherm plot (B) for Form B.

FIG. 13 shows the DVS weight change plot (A) and the DVS isotherm plot (B) for Form C.

### DETAILED DESCRIPTION OF THE INVENTION

It has been discovered that the hydrobromide salt of linaprazan glurate under certain conditions may form stable crystalline forms (polymorphs), having high crystallinity and high chemical stability. The new polymorphs are therefore expected to be useful in pharmaceutical compositions of linaprazan glurate. In a first aspect, therefore, the invention relates to a crystalline HBr salt of linaprazan glurate.

In one embodiment, the invention provides a crystalline HBr salt of linaprazan glurate wherein the crystalline HBr salt is stable at a relative humidity (RH) of 94% at room temperature. Such crystalline HBr salts can be stable under these conditions for at least 1 day, 1 week, 1 month, 3 months, 6 months, 1 year, 2 years, 3 years or even longer.

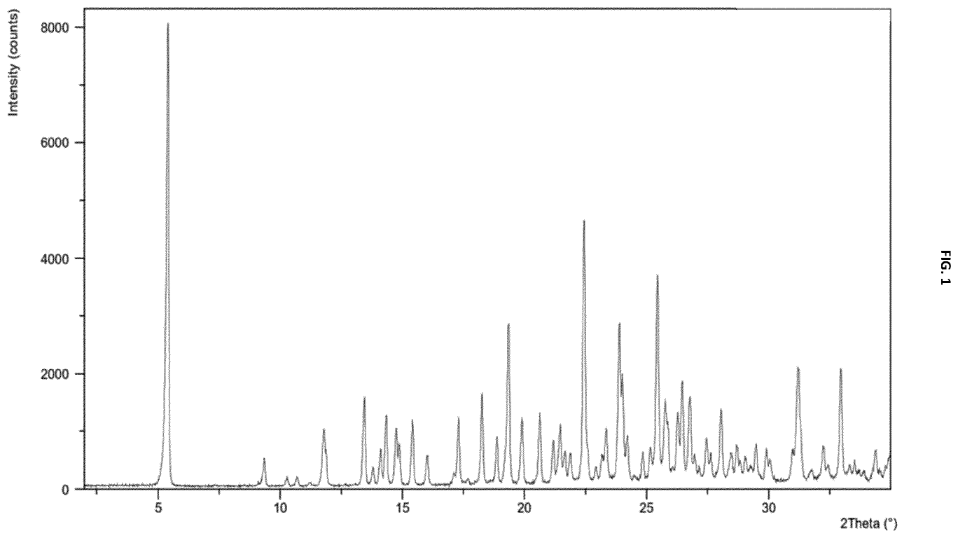

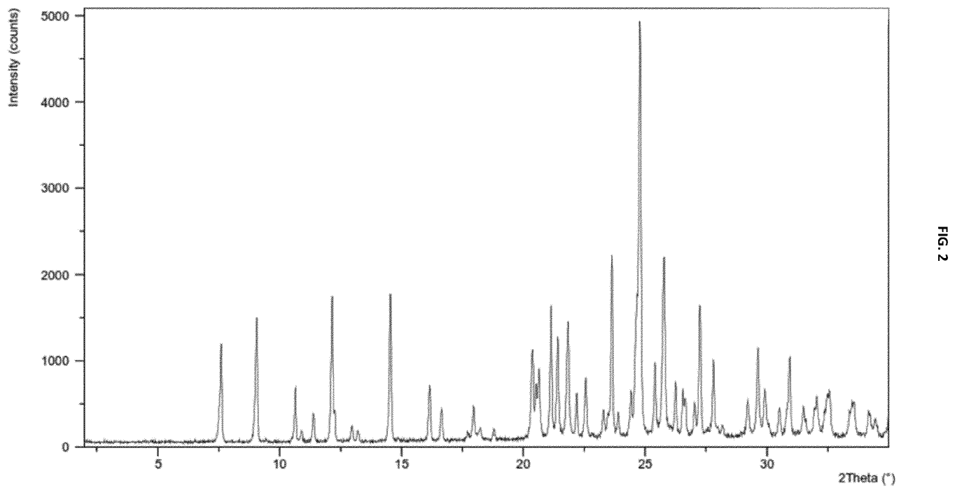

In some embodiments, the crystalline HBr salt is an anhydrate. In one embodiment, the crystalline anhydrate is Form A. This form may be prepared directly from the free base of linaprazan glurate, or by certain crystallisation techniques using the hydrobromide salt thereof, e.g. from a slurry in 1,4-dioxane; by anti-solvent crystallisation from MeOH, pyridine or DMF and certain anti-solvents; or by reversed crystallisation from DMF and MTBE. In one embodiment, Form A has an X-ray powder diffraction (XRPD) pattern, obtained with CuKα1-radiation, with at least two peaks at °2θ values selected from the list consisting of 5.4±0.2, 18.3±0.2, 19.4±0.2, 22.4±0.2, 23.9±0.2, 24.0 0.2, 25.4±0.2, 26.5±0.2, 31.2±0.2, and 32.9±0.2. In some embodiments, Form A has an XRPD pattern, obtained with CuKα1-radiation, with at least peaks at °2θ values of 5.4±0.2 and 19.4±0.2, or at ° 26 values of 5.4±0.2 and 22.4±0.2, or at °2θ values of 5.4±0.2 and 25.4±0.2, or at °2θ values of 19.4±0.2 and 22.4±0.2, or at °2θ values of 19.4±0.2 and 25.4±0.2, or at °2θ values of 22.4±0.2 and 25.4±0.2. In some embodiments, Form A has an XRPD pattern, obtained with CuKα1-radiation, with at least four peaks at 026 values selected from the list consisting of 5.4±0.2, 18.3±0.2, 19.4±0.2, 22.4±0.2, 23.9±0.2, 24.0±0.2, 25.4±0.2, 26.5±0.2, 31.2±0.2, and 32.9±0.2. In some embodiments, Form A has an XRPD pattern, obtained with CuKα1-radiation, with at least peaks at °2θ values of 5.4±0.2, 19.4±0.2, 22.4±0.2 and 25.4±0.2. In some embodiments, Form A has an XRPD pattern, obtained with CuKα1-radiation, with at least peaks at °2θ values of 5.4±0.2, 19.4±0.2, 22.4±0.2 and 25.4±0.2, and one or more of 18.3±0.2, 23.9±0.2, 24.0±0.2, 26.5±0.2, 31.2±0.2 and 32.9±0.2. In some embodiments, Form A has an XRPD pattern, obtained with CuKα1-radiation, with at least peaks at °2θ values of 5.4±0.2, 19.4±0.2, 22.4±0.2, 23.9±0.2, 25.4±0.2, 31.2±0.2 and 32.9±0.2. In some embodiments, Form A has an XRPD pattern, obtained with CuKα1-radiation, with at least peaks at °2θ values of 5.4±0.2, 18.3±0.2, 19.4±0.2, 22.4±0.2, 23.9±0.2, 24.0±0.2, 25.4±0.2, 26.5±0.2, 31.2±0.2, and 32.9±0.2. In some embodiments, Form A has an XRPD pattern, obtained with CuKα1-radiation, with at least peaks at 026 values of 5.4±0.2, 18.3±0.2, 19.4±0.2, 22.4±0.2, 23.9±0.2, 24.0 0.2, 25.4±0.2, 26.5±0.2, 31.2±0.2, and 32.9±0.2, and one or more of 13.5±0.2, 14.4±0.2, 20.6±0.2, 25.8±0.2 and 26.8±0.2. In a particular embodiment, the invention relates to Form A, having an XRPD pattern, obtained with CuKα1-radiation, substantially as shown in FIG. 1. In a further embodiment, the invention relates to Form A, having an XRPD pattern obtained with CuKα1-radiation, with peaks as shown in table 8.

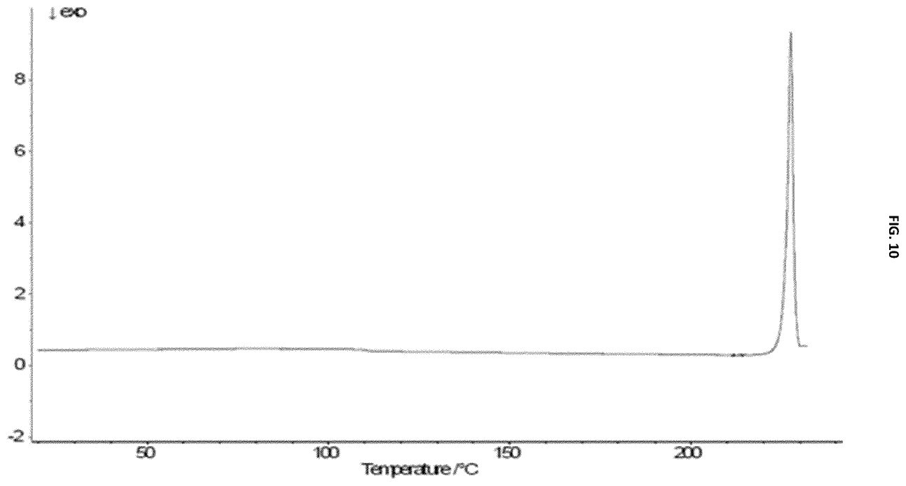

In some embodiments, Form A has a DSC curve comprising an endotherm between about 194° C. and about 198° C., such as at about 196° C. In a particular embodiment, Form A has a DSC curve comprising an endotherm between about 194° C. and about 198° C., such as at about 196° C., followed by an additional endotherm between about 220° C. and about 224° C., such as at about 222° C. The DSC curve of Form A is shown in FIG. 8.

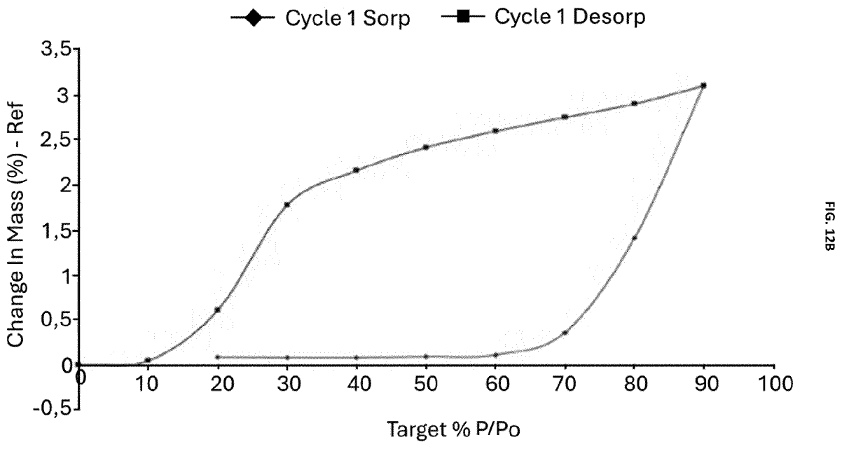

Dynamic vapour sorption analysis has shown that Form A has a very low hygroscopicity, with a water uptake of about 0.15% at 80% RH. This low hygroscopicity is considered advantageous, as the water content of the crystals remains substantially constant even with humidity changes within the normal relative humidity range of about 30% to about 80% RH. In some embodiments, Form A is stable at a relative humidity up to 90% at a temperature of 25° C. The DVS plot of Form A is shown in FIG. 11.

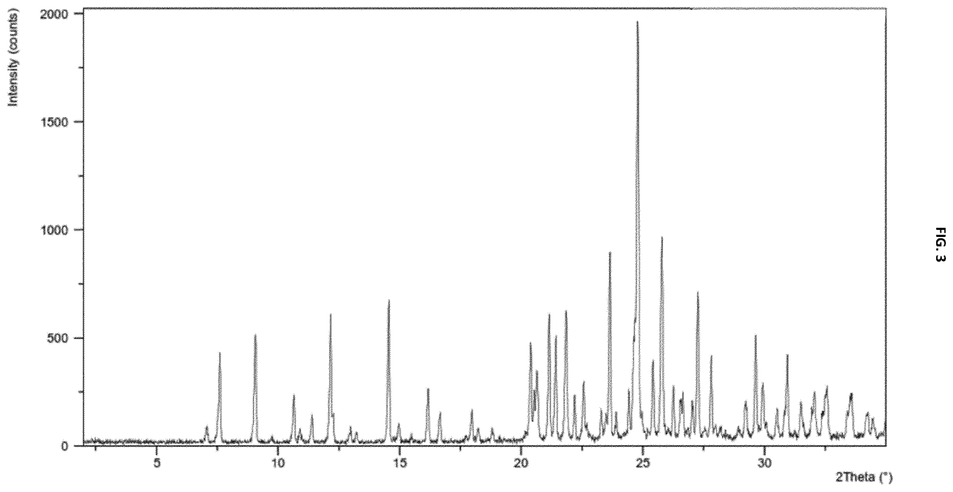

In another embodiment, the crystalline HBr salt is Form B. This form may be prepared from certain crystallisation techniques using the hydrobromide salt of X842, e.g. from a slurry in MeOH or a mixture of methanol and toluene; by evaporation from ethanol; by reversed anti-solvent crystallisation from benzyl alcohol and isopropyl acetate or MTBE, or from pyridine and MTBE; or by cooling from methanol. In one embodiment, Form B has an X-ray powder diffraction (XRPD) pattern, obtained with CuKα1-radiation, with at least two peaks at °2θ values selected from the list consisting of 9.1±0.2, 12.2±0.2, 14.5±0.2, 21.1±0.2, 21.4±0.2, 21.8±0.2, 23.6±0.2, 24.8±0.2, 25.8±0.2, and 27.3±0.2. In some embodiments, Form B has an XRPD pattern, obtained with CuKα1-radiation, with at least peaks at °2θ values of 23.6±0.2 and 24.8±0.2, or at °2θ values of 24.8±0.2 and 25.8±0.2. In some embodiments, Form B has an XRPD pattern, obtained with CuKα1-radiation, with at least four peaks at °2θ values selected from the list consisting of 9.1±0.2, 12.2±0.2, 14.5±0.2, 21.1±0.2, 21.4±0.2, 21.8±0.2, 23.6±0.2, 24.8±0.2, 25.8±0.2, and 27.3±0.2. In some embodiments, Form B has an XRPD pattern, obtained with CuKα1-radiation, with at least peaks at 026 values of 14.5±0.2, 23.6±0.2, 24.8±0.2 and 25.8±0.2. In some embodiments, Form B has an XRPD pattern, obtained with CuKα1-radiation, with at least peaks at 026 values of 14.5±0.2, 23.6±0.2, 24.8±0.2 and 25.8±0.2, and one or more of 9.1±0.2, 12.2±0.2, 21.1±0.2, 21.4±0.2, 21.8±0.2 and 27.3±0.2. In some embodiments, Form B has an XRPD pattern, obtained with CuKα1-radiation, with at least peaks at 026 values of 12.1±0.2 14.5±0.2, 21.1±0.2, 23.6±0.2, 24.8±0.2, 25.8±0.2 and 27.3±0.2. In some embodiments, Form B has an XRPD pattern, obtained with CuKα1-radiation, with at least peaks at 026 values of 9.1±0.2, 12.2±0.2, 14.5±0.2, 21.1±0.2, 21.4±0.2, 21.8±0.2, 23.6±0.2, 24.8±0.2, 25.8±0.2, and 27.3±0.2. In some embodiments, Form B has an XRPD pattern, obtained with CuKα1-radiation, with at least peaks at 026 values of 9.1±0.2, 12.2±0.2, 14.5±0.2, 21.1±0.2, 21.4±0.2, 21.8±0.2, 23.6±0.2, 24.8±0.2, 25.8±0.2, and 27.3±0.2, and one or more of 7.6±0.2, 20.4±0.2, 27.8±0.2, 29.6±0.2 and 30.9±0.2. In a particular embodiment, the invention relates to Form B, having an XRPD pattern, obtained with CuKα1-radiation, substantially as shown in FIG. 2. In a further embodiment, the invention relates to Form B, having an XRPD pattern obtained with CuKα1-radiation, with peaks as shown in table 9.

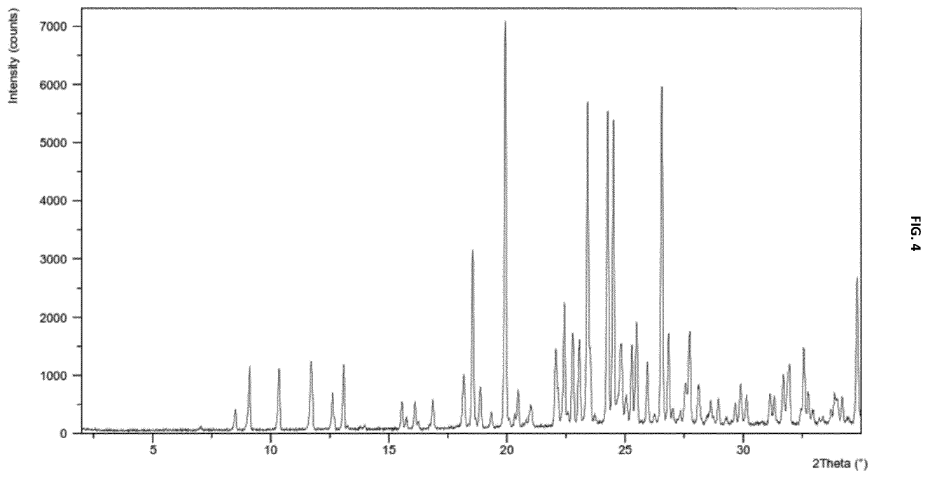

It was found that the X-ray diffractogram of Form B sometimes contained a few additional peaks, for instance in experiments where Form B was formed in methanol or methanol mixtures, in acetonitrile, or by evaporation from ethanol. The additional peaks are believed to belong to small amounts of a presumed channel solvate. In some embodiments, therefore, the invention relates to Form B, having an XRPD pattern, obtained with CuKα1-radiation, substantially as shown in FIG. 3. The channel solvate seems to transform into Form B upon drying.

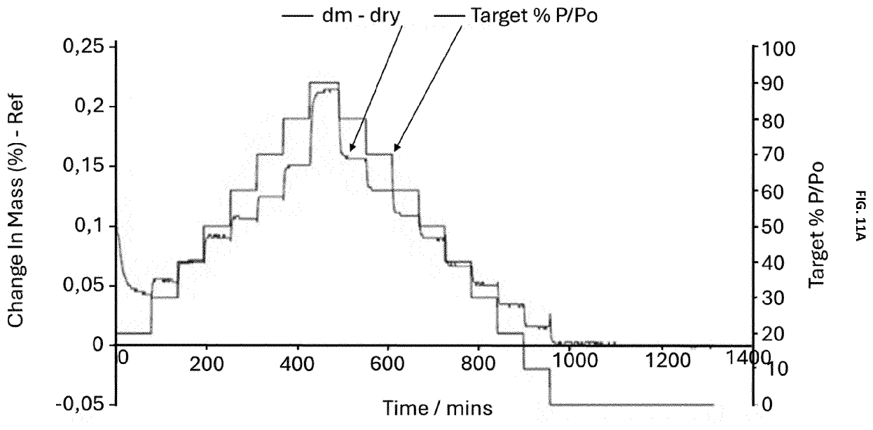

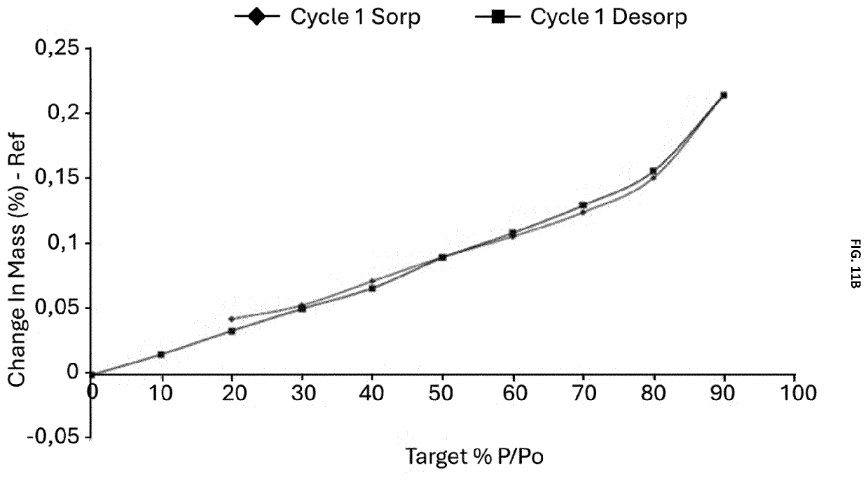

In some embodiments, Form B has a DSC curve comprising an endotherm between about 115° C. and about 121° C., such as at about 118° C. In a particular embodiment, Form B has a DSC curve comprising an endotherm between about 115° C. and about 121° C., such as at about 118° C., followed by an additional endotherm between about 202° C. and about 206° C., such as at about 204° C. The DSC curve of Form B is shown in FIG. 9.

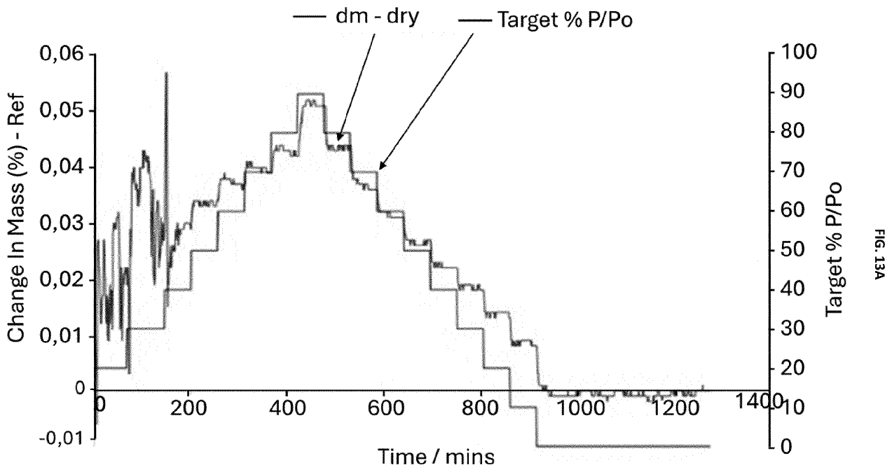

It has been found that the water content of Form B can vary between about 0 and 3%, depending on the relative humidity, as shown in FIG. 12. The significant moisture interaction of Form B indicates that it forms a hydrate at elevated humidity (where about 3% corresponds to a monohydrate), and returns back to an anhydrate when dried.

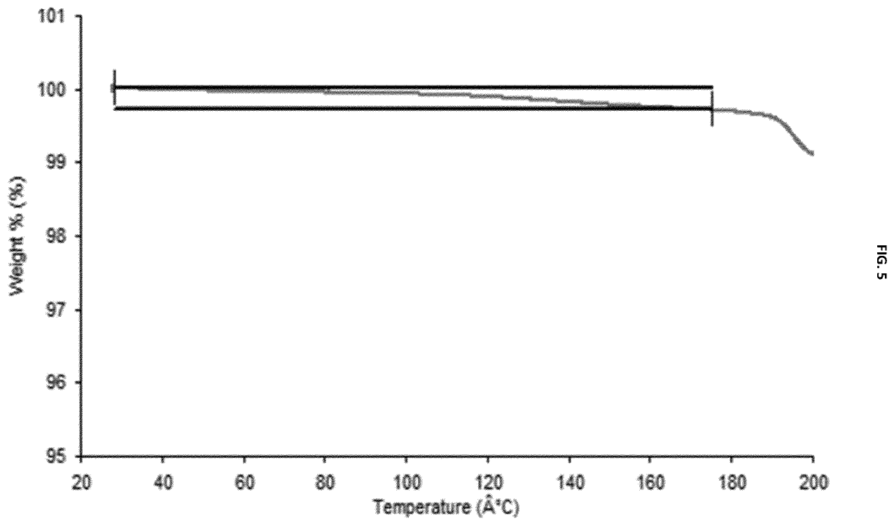

In a further embodiment, the crystalline anhydrate is Form C. This form may also be prepared by certain crystallisation techniques using the hydrobromide salt, e.g. from a slurry in EtOH, or mixtures of DMF/toluene, benzyl alcohol/toluene, DMF/isopropyl acetate or EtOH/water; by anti-solvent crystallisation from DMF or benzyl alcohol together with certain anti-solvents; or by reversed anti-solvent crystallisation from DMF and acetone. In one embodiment, Form C has an X-ray powder diffraction (XRPD) pattern, obtained with CuKα1-radiation, with at least two peaks at °2θ values selected from the list consisting of 18.6±0.2, 19.9±0.2, 22.4±0.2, 22.8±0.2, 23.4±0.2, 24.3±0.2, 24.5±0.2, 25.5±0.2, 26.6±0.2, and 34.8±0.2. In some embodiments, Form C has an XRPD pattern, obtained with CuKα1-radiation, with at least peaks at °2θ values of 19.9±0.2 and 23.4±0.2, or at ° 26 values of 19.9±0.2 and 26.6±0.2. In some embodiments, Form C has an XRPD pattern, obtained with CuKα1-radiation, with at least four peaks at °2θ values selected from the list consisting of 18.6±0.2, 19.9±0.2, 22.4±0.2, 22.8±0.2, 23.4±0.2, 24.3±0.2, 24.5±0.2, 25.5±0.2, 26.6±0.2, and 34.8±0.2. In some embodiments, Form C has an XRPD pattern, obtained with CuKα1-radiation, with at least peaks at 026 values of 19.9±0.2, 23.4±0.2, 24.5±0.2 and 26.6±0.2. In some embodiments, Form C has an XRPD pattern, obtained with CuKα1-radiation, with at least peaks at 026 values of 19.9±0.2, 23.4±0.2, 24.5±0.2 and 26.6±0.2, and one or more of 18.6±0.2, 22.4±0.2, 22.8±0.2, 24.3±0.2, 25.5±0.2, and 34.8±0.2. In some embodiments, Form C has an XRPD pattern, obtained with CuKα1-radiation, with at least peaks at 026 values of 18.6±0.2, 19.9±0.2, 23.4±0.2, 24.3±0.2, 24.5±0.2, 26.6±0.2 and 34.8±0.2. In some embodiments, Form C has an XRPD pattern, obtained with CuKα1-radiation, with at least peaks at 026 values of 18.6±0.2, 19.9±0.2, 22.4±0.2, 22.8±0.2, 23.4±0.2, 24.3±0.2, 24.5±0.2, 25.5±0.2, 26.6±0.2, and 34.8±0.2. In some embodiments, Form C has an XRPD pattern, obtained with CuKα1-radiation, with at least peaks at 026 values of 18.6±0.2, 19.9±0.2, 22.4±0.2, 22.8±0.2, 23.4±0.2, 24.3±0.2, 24.5±0.2, 25.5±0.2, 26.6±0.2, and 34.8±0.2, and one or more of 23.1±0.2, 24.8±0.2, 25.3±0.2, 26.9±0.2 and 27.7±0.2. In a particular embodiment, the invention relates to Form C, having an XRPD pattern, obtained with CuKα1-radiation, substantially as shown in FIG. 4. In a further embodiment, the invention relates to Form C, having an XRPD pattern obtained with CuKα1-radiation, with peaks as shown in table 10.

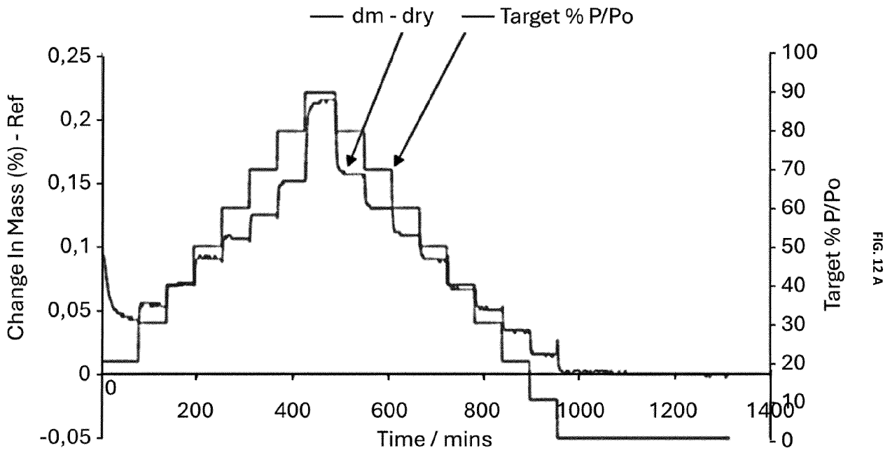

In some embodiments, Form C has a DSC curve comprising an endotherm between about 214° C. and about 230° C., such as at about 226° C., shown in FIG. 10.

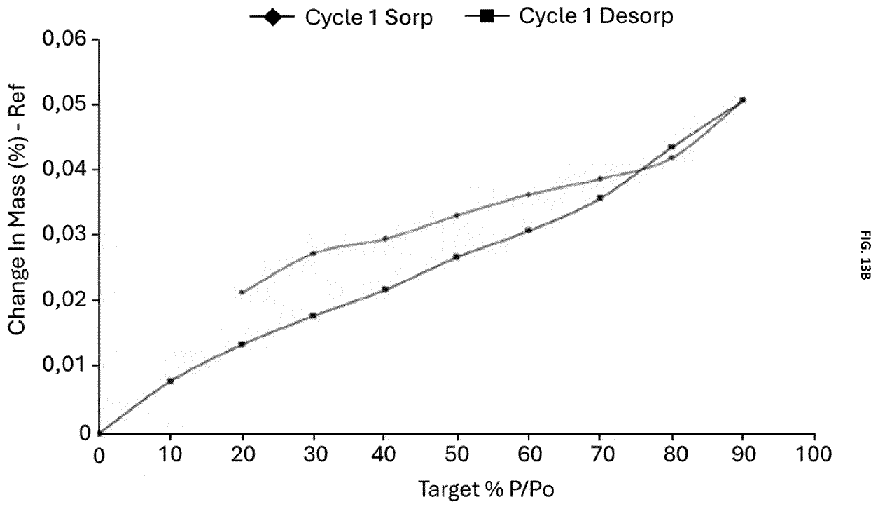

Dynamic vapor sorption analysis has shown that the humidity interaction of Form C is very low, with a water content of not more than about 0.05% at 90% RH. The DVS plot of Form C is shown in FIG. 13.

In one embodiment, the invention relates to a crystalline HBr salt of linaprazan glurate having a crystallinity of greater than 99%.

In a second aspect, the invention relates to a pharmaceutical composition comprising a therapeutically effective amount of a crystalline HBr salt of linaprazan glurate as disclosed herein, in association with one or more pharmaceutically acceptable excipients. The excipients may e.g. include fillers, binders, surfactants, disintegrants, glidants and lubricants. In some embodiments, the crystalline HBr salt of linaprazan glurate is Form A. In some embodiments, the crystalline HBr salt of linaprazan glurate is Form B. In some embodiments, the crystalline HBr salt of linaprazan glurate is Form C.

In some embodiments, the pharmaceutical composition comprises a crystalline HBr salt of linaprazan glurate, such as Form A, Form B or Form C, having a polymorphic purity of at least about 90%. In some embodiments, the polymorphic purity is at least about 95%. In some embodiments, the polymorphic purity is at least about 98%. For example, the polymorphic purity may be at least about 98.5%, such as at least about 99%, such as at least about 99.5%, such as at least about 99.8%, or such as at least about 99.9%. In some embodiments, a pharmaceutical composition comprising a crystalline HBr salt of linaprazan glurate is substantially free of other forms of linaprazan glurate. For example, in some embodiments, a pharmaceutical composition comprising Form A is substantially free of other forms of linaprazan glurate, such as Form B or Form C of linaprazan glurate. In some embodiments, Form A contains less than about 15% by weight of Form B, Form C, or any other polymorph of linaprazan glurate. For example, Form A contains less than about 14%, about 13%, about 12%, about 11%, about 10%, about 9%, about 8%, about 7%, about 6%, about 5%, about 4%, about 3%, about 2%, about 1% or less by weight of Form B, Form C, or any other polymorph of linaprazan glurate. In some embodiments, Form B contains less than about 15% by weight of Form A, Form C, or any other polymorph of linaprazan glurate. For example, Form B contains less than about 14%, about 13%, about 12%, about 11%, about 10%, about 9%, about 8%, about 7%, about 6%, about 5%, about 4%, about 3%, about 2%, about 1% or less by weight of Form A, Form C, or any other polymorph of linaprazan glurate. In some embodiments, Form C contains less than about 15% by weight of Form A, Form B, or any other polymorph of linaprazan glurate. For example, Form C contains less than about 14%, about 13%, about 12%, about 11%, about 10%, about 9%, about 8%, about 7%, about 6%, about 5%, about 4%, about 3%, about 2%, about 1% or less by weight of Form A, Form B, or any other polymorph of linaprazan glurate.

In some embodiments, the pharmaceutical composition can comprise between about 1% and about 100%, such as between about 1% and about 50%, or such as between about 1% and about 20% by weight of a crystalline HBr salt of linaprazan glurate. For example, the composition can comprise between about 1% and about 15%, or between about 5% and about 20%, such as between about 1% and about 10%, between about 5% and about 15%, and between about 10% and about 20%, or such as between about 1% and about 5%, between about 5% and about 10%, between about 10% and about 15%, and between about 15% and about 20% by weight of a crystalline HBr salt of linaprazan glurate. In some embodiments, the composition comprises about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1% by weight of a crystalline HBr salt of linaprazan glurate.

In some embodiments, the composition comprises a unit dose of about 25 mg to about 150 mg of a crystalline HBr salt of linaprazan glurate. For example, the composition can comprise between about 25 mg and about 50 mg, between about 50 mg and about 75 mg, between about 75 mg and about 100 mg, between about 100 mg and about 125 mg, or between about 125 mg and about 150 mg. In some embodiments, the composition comprises about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg of a crystalline HBr salt of linaprazan glurate. The daily dose can be administered as a single dose or divided into two, three, or more unit doses.

In some embodiments, the pharmaceutical composition comprises a surfactant. The surfactant may be a cationic surfactant, an anionic surfactant or a nonionic surfactant. Examples of cationic surfactants include, but are not limited to, cetyltrimethylammonium bromide (cetrimonium bromide) and cetylpyridinium chloride. Examples of anionic surfactants include, but are not limited to, sodium dodecyl sulfate (sodium lauryl sulfate) and ammonium dodecyl sulfate (ammonium lauryl sulfate). Examples of nonionic surfactants include, but are not limited to, glycerol monooleate, glycerol monostearate, polyoxyl castor oil (Cremophor EL), poloxamers (e.g., poloxamer 407 or 188), polysorbate 80 and sorbitan esters (Tween).

In some embodiments, the pharmaceutical composition comprises a filler. Examples of suitable fillers include, but are not limited to, dicalcium phosphate dihydrate, calcium sulfate, lactose (such as lactose monohydrate), sucrose, mannitol, sorbitol, cellulose, microcrystalline cellulose, dry starch, hydrolyzed starches and pregelatinized starch.

In some embodiments, the pharmaceutical composition comprises a binder. Examples of suitable binders include, but are not limited to, starch, pregelatinized starch, gelatin, sugars (such as sucrose, glucose, dextrose, lactose and sorbitol), polyethylene glycol, waxes, natural and synthetic gums (such as acacia gum and tragacanth gum), sodium alginate, cellulose derivatives (such as hydroxypropylmethylcellulose (or hypromellose), hydroxypropylcellulose and ethylcellulose) and synthetic polymers (such as acrylic acid and methacrylic acid copolymers, methacrylic acid copolymers, methyl methacrylate copolymers, aminoalkyl methacrylate copolymers, polyacrylic acid/polymethacrylic acid copolymers and polyvinylpyrrolidone (povidone)).

In some embodiments, the pharmaceutical composition comprises a disintegrant. Examples of suitable disintegrants include, but are not limited to, dry starch, modified starch (such as (partially) pregelatinized starch, sodium starch glycolate and sodium carboxymethyl starch), alginic acid, cellulose derivatives (such as sodium carboxymethylcellulose, hydroxypropyl cellulose, and low substituted hydroxypropyl cellulose (L-HPC)) and cross-linked polymers (such as carmellose, croscarmellose sodium, carmellose calcium and cross-linked PVP (crospovidone)).

In some embodiments, the pharmaceutical composition comprises a glidant or lubricant. Examples of suitable glidants and lubricants include, but are not limited to, talc, magnesium stearate, calcium stearate, sodium stearyl fumarate, stearic acid, glyceryl behenate, colloidal anhydrous silica, aqueous silicon dioxide, synthetic magnesium silicate, fine granulated silicon oxide, starch, sodium lauryl sulfate, boric acid, magnesium oxide, waxes (such as carnauba wax), hydrogenated oil, polyethylene glycol, sodium benzoate, polyethylene glycol, and mineral oil.

In general, pharmaceutical compositions may be prepared in a conventional manner using conventional excipients. In some embodiments, the ingredients of the formulation are mixed to a homogenous mixture and then formulated as tablets or capsules. The homogenous mixture of the ingredients may be compressed into tablets using conventional techniques, such as a rotary tablet press. The mixture of ingredients may also be granulated. For instance, the mixture of ingredients may be wetted by the addition of a liquid, such as water and/or an appropriate organic solvent (e.g., ethanol or isopropanol), and thereafter granulated and dried. Alternatively, granules may be prepared by dry granulation, such as by roller compaction. The granules obtained may be compressed into tablets using conventional techniques. Capsules may comprise a powder mixture or small multiparticulates (such as granules, extruded pellets or minitablets) of the ingredients. If desirable, any of the tablets, capsules, granules, extruded pellets and minitablets mentioned above may be coated with one or more coating layers. Such coating layers may be applied by methods known in the art, such as by film coating involving perforated pans and fluidized beds. In some embodiments, the formulation is in the form of a tablet.

Following absorption into the blood stream, linaprazan glurate is quickly metabolized into linaprazan, which is the active metabolite. Whereas the plasma concentration of linaprazan glurate is only very low and difficult to determine, the plasma concentration of linaprazan may be determined instead. Phase I studies have indicated that certain doses of linaprazan glurate should be able to maintain the intra-gastric pH above 4 for 24 hours after administration. It is estimated that this requires a minimal plasma concentration (Cₘᵢₙ) of linaprazan of at least about 240 nmol/L after 22 hours. At such doses, a once daily oral administration of the formulation would be sufficient. In some embodiments, therefore, a single unit dose of a pharmaceutical composition of linaprazan glurate provides a Cₘᵢₙ of linaprazan in a human of at least about 240 nmol/L after 22 hours following oral administration of the pharmaceutical composition to said human. In other embodiments, a daily administration of two unit doses of a pharmaceutical composition of linaprazan glurate provides a Cₘᵢₙ of linaprazan in a human of at least about 240 nmol/L after 10 hours following oral administration of the last unit dose of the pharmaceutical composition to said human.

In one aspect, the invention relates to a crystalline HBr salt of linaprazan glurate, for use in therapy.

The crystalline forms of the HBr salt of linaprazan glurate disclosed herein can be used in the treatment or prevention of diseases or conditions wherein inhibition of gastric acid secretion is necessary or desirable, such as in H. pylori eradication. Examples of such diseases and conditions include gastrointestinal inflammatory diseases and gastric acid related diseases, such as gastritis, gastroesophageal reflux disease (GERD), erosive gastroesophageal reflux disease (eGERD), H. pylori infection, Zollinger-Ellison syndrome, peptic ulcer disease (including gastric ulcers and duodenal ulcers), bleeding gastric ulcer, symptoms of gastroesophageal reflux disease (including heartburn, regurgitation and nausea), gastrinoma and acute upper gastrointestinal bleeding.

In one aspect, the invention relates to a pharmaceutical composition comprising a therapeutically effective amount of a crystalline HBr salt of linaprazan glurate, as disclosed herein, for use in the treatment or prevention of a gastrointestinal inflammatory disease or a gastric acid related disease.

In another aspect, the invention relates to a method for treating or preventing a gastrointestinal inflammatory disease or a gastric acid related disease in a subject in need thereof, comprising administering a pharmaceutical composition comprising a therapeutically effective amount of a crystalline form of the HBr salt of linaprazan glurate, as disclosed herein. In some embodiments, the crystalline form of the HBr salt of linaprazan glurate is Form A. In other embodiments, the crystalline form of the HBr salt of linaprazan glurate is Form B. In further embodiments, the crystalline form of the HBr salt of linaprazan glurate is Form C.

In some embodiments, the gastrointestinal inflammatory disease or the gastric acid related disease is erosive gastroesophageal reflux disease (eGERD).

In further embodiments, the treatment of GERD is on-demand treatment of GERD.

As used herein, the term “polymorph” refers to crystals of the same molecule that have different physical properties as a result of the order of the molecules in the crystal lattice. Polymorphs of a single compound have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bioavailability), solubility, melting point, chemical stability, physical stability, powder flowability, water sorption, compaction, and particle morphology. Differences in stability can result from changes in chemical reactivity (e.g. differential oxidation, such that a dosage form discolours more rapidly when comprised of one polymorph than when comprised of another polymorph) or mechanical changes (e.g., crystal changes on storage as a kinetically favoured polymorph converts to a thermodynamically more stable polymorph) or both (e.g., one polymorph is more hygroscopic than the other). As a result of solubility/dissolution differences, some transitions affect potency and/or toxicity. In addition, the physical properties of the crystal may be important in processing; for example, one polymorph might be more likely to form solvates or might be difficult to filter and wash free of impurities (i.e., particle shape and size distribution might be different between one polymorph relative to the other). “Polymorph” does not include amorphous forms of the compound.

As used herein, the term “amorphous” refers to a non-crystalline form of a compound which may be a solid state form of the compound or a solubilized form of the compound. For example, “amorphous” refers to a compound without a regularly repeating arrangement of molecules or external face planes.

As used herein, the term “anhydrate” or “anhydrous form” refers to a polymorph of linaprazan glurate that has 0.5% or less by weight water, for example 0.4% or less, or 0.3% or less, or 0.2% or less, or 0.1% or less by weight water.

As used herein, the term “anhydrate” refers to a polymorph of linaprazan glurate that has 0.5% or less by weight water, for example 0.4% or less, or 0.3% or less, or 0.2% or less, or 0.1% or less by weight water.

As used herein, the term “polymorphic purity” when used in reference to a composition comprising a polymorph of linaprazan glurate, refers to the percentage of one specific polymorph relative to another polymorph or an amorphous form of linaprazan glurate in the referenced composition. For example, a composition comprising Form A having a polymorphic purity of 90% would comprise 90 weight parts of Form A and 10 weight parts of other crystalline and/or amorphous forms of linaprazan glurate, such as Form B or Form C.

As used herein, the terms “effective amount” or “therapeutically effective amount” refer to a sufficient amount of linaprazan glurate that, following administration to a subject, will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic use is the amount of linaprazan glurate required to provide a clinically significant decrease in disease symptoms. An appropriate “effective” amount in any individual case is determined using any suitable technique, such as a dose escalation study.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “pharmaceutically acceptable” refers to those compounds, materials, compositions and/or dosage forms that are suitable for human pharmaceutical use and that are generally safe, non-toxic and neither biologically nor otherwise undesirable. As used herein, a compound or composition is “substantially free” of one or more other components if the compound or composition contains no significant amount of such other components. Such components can include starting materials, residual solvents, or any other impurities that can result from the preparation of and/or isolation of the compounds and compositions provided herein. In some embodiments, a polymorph form provided herein is “substantially free” from impurities. The purity of a particular polymorph is preferably greater than about 90% (w/w), such as greater than about 95% (w/w), such as greater than about 97% (w/w), or such as greater than about 99% (w/w). In some embodiments, the purity of a particular polymorph is greater than 99.5% (w/w), or even greater than 99.9% (w/w). In some embodiments, the impurity in a particular polymorph is less than about 1% (w/w), such as less than about 0.5% (w/w), or such as less than about 0.1% (w/w). The total amount of impurities may be determined e.g. by high-performance liquid chromatography (HPLC) methods.

In some embodiments, a polymorph form provided herein is substantially free of other polymorph forms. In some embodiments, a particular polymorph of linaprazan glurate is “substantially free” of other polymorphs if the particular polymorph constitutes at least about 95% by weight of linaprazan glurate present. In some embodiments, a particular polymorph of linaprazan glurate is “substantially free” of other polymorphs if the particular polymorph constitutes at least about 97%, about 98%, about 99%, or about 99.5% by weight of linaprazan glurate present.

As used herein, a compound is “substantially present” as a given polymorph if at least about 50% by weight of the compound is in the form of that polymorph, for example if at least about 60%, at least about 70%, at least about 80%, or at least about 90% by weight of the compound is in the form of that polymorph. In some embodiments, at least about 95%, such as at least about 96%, such as at least about 97%, such as at least about 98%, such as at least about 99% or such as at least about 99.5% by weight of the compound is in the form of that polymorph.

As used herein, the term “stable” means that the polymorphs do not exhibit a change in one or more of polymorph form (e.g., an increase or decrease of a certain form), appearance, pH, percent impurities, activity (as measured by in vitro assays), or osmolarity over time. In some embodiments, the polymorphs provided herein are stable for at least 1, 2, 3 or 4 weeks. For example, the polymorphs do not exhibit a change in one or more of polymorph form (e.g., an increase or decrease of a certain form), appearance, pH, percent impurities, activity (as measured by in vitro assays), or osmolarity over at least 1, 2, 3 or 4 weeks. In some embodiments, the polymorphs provided herein are stable for at least 1, 2, 3, 4, 5, 6, 7, 8, 9, 10, 11 or 12 months. For example, the polymorphs do not exhibit a change in one or more polymorph form (e.g., an increase or decrease of a certain form), appearance, pH, percent impurities, activity (as measured by in vitro assays), or osmolarity over at least 1, 2, 3, 4, 5, 6, 7, 8, 9, 10, 11 or 12 months. In the above, the phrase “do not exhibit a change” refers to a change of less than 5% (e.g., less than 4%, less than 3%, less than 2%, less than 1%) as measured for any of the parameters over the relevant time period.

The crystallinity of a polymorph of the HBr salt of linaprazan glurate may be measured e.g. by X-ray powder diffraction (XRPD) methods or by differential scanning calorimetry (DSC) methods. When reference is made herein to a crystalline compound, preferably the crystallinity is greater than about 70%, such as greater than about 80%, particularly greater than about 90%, more particularly greater than about 95%. In some embodiments, the degree of crystallinity is greater than about 98%. In some embodiments, the degree of crystallinity is greater than about 99%. The % crystallinity refers to the percentage by weight of the total sample mass which is crystalline.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

The invention will now be described by the following examples which do not limit the invention in any respect. All cited documents and references mentioned herein are incorporated by reference in their entireties.

Abbreviations

| | |
|---|---|
| DMF | N,N-dimethylformamide |
| DMSO | dimethyl sulfoxide |
| EtOAc | ethyl acetate |
| EtOH | ethanol |
| MeCN | acetonitrile |
| MeOH | methanol |
| MTBE | methyl tert-butyl ether |
| RH | relative humidity |

### EXPERIMENTAL METHODS

### General Methods

¹H-NMR spectra were recorded on a Bruker 400 MHz instrument at 25° C. and referenced to residual protic solvent in the deuterated solvent used: DMSO-d₆ (SH 2.50 ppm).

Analytical HPLC-MS was performed using an Agilent 1100 series Liquid Chromatography/Mass Selective Detector (MSD) (Single Quadrupole) equipped with an electrospray interface and a UV diode array detector. Analyses were performed using an ACE 3 C8 (3.0×50 mm) column with a gradient of acetonitrile in 0.1% aqueous TFA over 3 minutes and a flow rate of 1 mL/minute.

All solvents were dried by adding molecular sieves prior to preparing solutions, unless indicated otherwise.

### X-Ray Powder Diffraction (XRPD) Analysis

Analyses were performed on a PanAlytical X'Pert Pro diffractometer equipped with a Cu-anode (45 kV, 40 mA), a Kα-1 Johansson monochromator (1.540598 Å) and a Pixcel detector. The 2-theta range was 2-35°, using a scan speed of 0.03° or 0.10°/s and a step size of 0.013°. Slow spinning sample holders were used. The samples were smeared out on zero background wafers of Si, producing a flat powdered surface. In one measurement, the sample was covered with a plastic film to prevent solvate evaporation. The measurements were performed using a programmable incident divergency slit.

It is known in the art that an X-ray powder diffraction pattern may be obtained having one or more measurement errors depending on measurement conditions (such as equipment, sample preparation or machine used). In particular, it is generally known that intensities in an XRPD pattern may fluctuate depending on measurement conditions and sample preparation. For example, persons skilled in the art of XRPD will realize that the relative intensities of peaks may vary according to the orientation of the sample under the test and on the type and setting of the instrument used. The skilled person will also realize that the position of reflections can be affected by the precise height at which the sample sits in the diffractometer and the zero calibration of the diffractometer. The surface planarity of the sample may also have a small effect. Hence a person skilled in the art will appreciate that the diffraction pattern presented herein is not to be construed as absolute and any crystalline form that provides a powder diffraction pattern substantially identical to those disclosed herein fall within the scope of the present disclosure (for further information, see R. Jenkins and R. L. Snyder, “Introduction to X-ray powder diffractometry”, John Wiley & Sons, 1996).

Thermogravimetric analysis (TGA)

Analyses were performed on a PerkinElmer TGA8000 instrument. A few mg of sample was gently charged into open Pt-pans and analysed by weight in a flow of dry nitrogen gas (40 mL/min), to ensure an inert atmosphere. The sample was scanned from 25 to 200° C. using a continuous scan speed of 10° C./min.

### Differential Scanning Calorimetry (DSC)

Analyses were performed on a Netzsch DSC 204F1 instrument. A few mg of sample was gently charged, and weighed, into Al pans. A lid with pre-made pinhole was adapted and crimped onto the pan. Conventional DSC with a heating rate of 10° C./min was employed. Minimum temperature (start) was 0° C. and maximum temperature was 230° C.

### Gravimetric Vapour Sorption (GVS)

Analyses were performed on an SMS DVS Advantage instrument. A few mg of the substance was added into an Al pan and exposed to stepwise RH changes according to 0-90-20% in steps of 10% RH, using open loop mode. The experiments were performed using a gas flow rate of 200 mL/min and at 25° C. The dm/dt criteria applied was 0.001 weight-%/min during a 5-minute window, with a maximum allowed time of 150 minutes and a minimum allowed time of 50 minutes for all steps except for the step at 0% RH, which had no criteria but was set to 6 h.

### EXAMPLES

### Example 1

### Preparation of the Hydrobromide Salt of Linaprazan Glurate

Linaprazan glurate (0.500 g, 1.04 mmol) was suspended in 2-propanol (25 mL) at 22° C. Addition of 48% aqueous HBr (0.175 g, 1.04 mmol) produced a slurry, followed by complete dissolution. The solution was cooled in an ice-bath for 10 minutes, after which it was filtered through a P3 fritted glass filter funnel. The obtained solid was dried under vacuum. Yield: 92% (0.539 g; colourless powder); 100% purity according to LCMS.

¹H NMR (400 MHz, DMSO-d₆): δ 13.65 (s, 1H), 12.05 (s, 1H), 8.98 (t, J=5.6 Hz, 1H), 8.40 (d, J=1.2 Hz, 1H), 7.35 (d, J=1.3 Hz, 1H), 7.30-7.05 (m, 3H), 6.18 (s, 1H), 4.43 (d, J=3.8 Hz, 2H), 4.22 (t, J=5.7 Hz, 2H), 3.59 (q, J=5.7 Hz, 2H), 2.51-2.30 (m, 11H), 2.24 (t, J=7.4 Hz, 2H), 1.74 (p, J=7.4 Hz, 2H). MS: (ESI+) m/z 481 (M+H).

### Example 2

### Polymorph Screen

A polymorph screen was performed on the HBr salt of linaprazan glurate to determine solubility, polymorphism and thermodynamic stability.

X-ray powder diffraction (XRPD), thermogravimetric analysis (TGA) and differential scanning calorimetry (DSC) indicated that the drug substance used for the screen was Form A. Prior to the crystallisation experiments, the solubility of the drug substance was determined in >20 solvents and solvent mixtures.

Slurry experiments:

Slurry experiments were performed in various solvents wherein the HBr salt of linaprazan glurate was found to have an intermediate solubility. About 10 to 220 mg of the drug substance was slurried in 14 different solvents (pure and binary solvents) at room temperature and at 40° C. for 2 weeks, unless indicated otherwise. The solid phase was isolated and analysed with XRPD. Table 1 summarizes the slurry experiments and indicates which solid form was obtained.

TABLE 1
Results of slurry experiments

| | | Solvent | | |
|---|---|---|---|---|
| Solvent | Salt (mg) | volume (mL) | Temperature | Solid state form |
| MeOH | 84.1 | 0.5 | RT | Form B |
| MeOH | 83.4 | 0.25 | 40° C. | Form B |
| EtOH | 16.7 | 1 | RT | Form C |
| EtOH | 35.7 | 1 | 40° C. | Form C |
| DMF/toluene 1:1 | 137.8 | 0.5 | RT | Form C |
| DMF/toluene 1:1 | 136.1 | 0.25 | 40° C. | Form C |
| MeOH/Toluene 4:1 | 93.2 | 0.5 | RT | Form B |
| MeOH/Toluene 4:1 | 92.1 | 0.25 | 40° C. | Form B* |
| Pyridine/water 1:1 | 56.6 | 1 | RT | Crystalline base form |
| Pyridine/water 1:1 | 56.9 | 0.5 | 40° C. | Crystalline base form |
| Benzyl alcohol/toluene 3:2 | 102.1 | 0.5 | RT | Form C |
| Benzyl alcohol/toluene 3:2 | 103.8 | 0.25 | 40° C. | Form C |
| DMSO/EtOAc 1:4 | 56.5 | 0.5 | RT | DMSO solvate |
| DMSO/EtOAc 1:4 | 122.8 | 0.5 | 40° C. | DMSO solvate |
| DMF/isopropyl acetate 2:3 | 41.2 | 1 | RT | Form C |
| DMF/isopropyl acetate 2:3 | 40.1 | 0.5 | 40° C. | Form C |
| EtOH/water 4:1 | 49.5 | 0.5 | RT | Form C** |
| EtOH/water 4:1 | 97.0 | 0.5 | 40° C. | Form C** |
| MeOH/water 4:1 | 125.2 | 0.5 | RT | Form B with a little amount |
| | | | | of channel solvate** |
| MeOH/water 4:1 | 125.1 | 0.25 | 40° C. | Form B with a little amount |
| | | | | of channel solvate** |
| DMSO/formamide 4:1 | 198.4 | 0.5 | RT | Unstable solvate** |
| DMSO/formamide 4:1 | 218.2 | 0.5 | 40° C. | Unstable solvate** |
| MeCN | 11.1 | 1 | RT | Form B with a little amount |
| | | | | of channel solvate** |
| MeCN | 20.2 | 1 | 40° C. | Form B with a little amount |
| | | | | of channel solvate** |
| Water | 11.6 | 1 | RT | Predominantly Form C with |
| | | | | traces of a free base form** |
| Water | 21.9 | 1 | 40° C. | Predominantly Form C with |
| | | | | traces of a free base form** |
| 1,4-dioxane | 9.7 | 2 | RT | Form A** |
| 1,4-dioxane | 19.85 | 2 | 40° C. | Form A** |

*analysed after 2, 3 and 4 weeks. Predominantly Form B at 2 weeks, which gradually turned into a channel solvate.
**analysed after 5 or 6 days.

Evaporation experiments:

Experiments were performed in seven solvents wherein the HBr salt of linaprazan glurate was found to have sufficiently high solubility. Approximately 10 mg of the drug substance was dissolved and left to evaporate slowly in a vial at RT, at 10° C. or at 50° C. The results are shown below in table 2.

TABLE 2
Results of evaporation experiments

| | | Solvent | | | |
|---|---|---|---|---|---|
| Solvent | Salt (mg) | volume (mL) | Temperature | | Solid state form |
| MeOH | 10.6 | 1 | RT | | Form B and crystalline base form |
| MeOH | 10.6 | 1 | RT | | Form B and crystalline base form |
| Formamide | 9.9 | 1 | RT | | Crystalline base form |
| DMF | 10.5 | 1 | RT | | Form C and crystalline base form |
| Pyridine | 10 | 0.5 | RT | | Amorphous |
| DMSO | 10.1 | 1 | RT then 50° C. | | No solid formed |
| | | | after 4 weeks | | |
| Benzyl | 9.8 | 1 | RT then 50° C. | | No solid formed |
| alcohol | | | after 4 weeks | | |
| EtOH | 9.9 | 5 | RT | | Form B |
| MeOH | 9.9 | 2.5 | 50° | C. | Amorphous |
| MeOH | 9.6 | 2.5 | 50° | C.* | Crystalline base form and |
| | | | | | amorphous |
| DMF | 9.7 | 1 | 50° | C. | Predominantly Form C with a little |
| | | | | | crystalline base form |
| Pyridine | 9.9 | 0.5 | 50° | C. | Predominantly Form B mixed with |
| | | | | | an unknown form |
| Pyridine | 9.9 | 0.5 | 10° | C. | Crystalline base form |
| EtOH | 9.7 | 5 | 50° | C. | Form B and channel solvate |

*vial covered with adhesive aluminium tape with one pinhole.

Anti-solvent crystallisation experiments at RT:

Crystallisations were performed from five solvents wherein the HBr salt of linaprazan glurate was found to have a high solubility, together with five anti-solvents wherein the HBr salt of linaprazan glurate has low solubility. The drug substance was dissolved in solvent 1 at RT. The solutions were then heated to 40° C. before letting them cool down to RT again. Thereafter, solvent 2 was added in 0.5 mL portions. The samples were analysed after 6 days. The solid phase was separated by vacuum filtration and analysed by XRPD. The crystalline solid forms that were obtained in the experiments are shown in Table 3.

TABLE 3
Results of antisolvent crystallisations

| | | Solvent 1 | | Solvent 2 | |
|---|---|---|---|---|---|
| Solvent 1 | Salt (mg) | volume (mL) | Solvent 2 | volume (mL) | Solid state form |
| MeOH | 19.4 | 2 | Water | 4 | Crystalline base form |
| MeOH | 20.8 | 2 | Acetone | 5 | Too little solid to analyse* |
| MeOH | 20.6 | 2 | MTBE | 4 | Form A |
| MeOH | 20.0 | 2 | Toluene | 5 | Too little solid to analyse* |
| DMF | 50.8 | 2 | Water | 2.5 | Crystalline base form |
| DMF | 50.5 | 1 | Acetone | 2.5 | Form A* |
| DMF | 49.6 | 1 | MTBE | 2.5 | Form C |
| DMF | 50.6 | 1 | Toluene | 2.5 | No solid* |
| Benzyl alcohol | 50.2 | 1 | Isopropyl | 2.5 | Form C |
| | | | acetate | | |
| Benzyl alcohol | 50.6 | 1 | Acetone | 2.5 | Form C* |
| Benzyl alcohol | 49.9 | 1 | MTBE | 2 | Form A and Form C as |
| | | | | | separate crystals |
| Benzyl alcohol | 50.8 | 1 | Toluene | 2.5 | Form C** |
| Pyridine | 50.2 | 1 | Water | 2.5 | Crystalline base form |
| Pyridine | 50.2 | 1 | Acetone | 2.5 | Crystalline base form** |
| Pyridine | 50.9 | 1 | MTBE | 2 | Form A |
| Pyridine | 49.8 | 1 | Toluene | 2.5 | Form C with a little Form A |
| DMSO | 50.0 | 1 | Water | 2 | Form A and crystalline base |
| | | | | | form |
| DMSO | 50.1 | 1 | Acetone | 2.5 | No solid* |
| DMSO | 50.2 | 1 | Toluene | 2 | No solid* |

*sample put in fridge after 6 days; piece of metal wire added after 2 weeks; sample put in freezer after 2.5 weeks.
**sample put in fridge after 6 days; analysed after 2 weeks.

### Cooled Anti-Solvent Crystallisation Experiments at 5° C.

Cooling experiments were performed with five solvents where the X842 HBr salt has a high solubility together with five solvents where X842 HBr salt has a low solubility. Samples were prepared by dissolving the drug substance in solvent 1, after which the solution was put in a 5° C. refrigerator. All solutions were then filtered through a 0.2 μm syringe filter, after which solvent 2 was added, pre-cooled to 5° C. The samples were analysed after 2 weeks, unless indicated otherwise. The solid phase was separated by vacuum filtration and analysed by XRPD. Crystalline solid forms obtained in the experiments are shown below in Table 4.

TABLE 4
Results of cooled anti-solvent experiments

| | | Solvent 1 | | Solvent 2 | |
|---|---|---|---|---|---|
| Solvent 1 | Salt (mg) | volume (mL) | Solvent 2 | volume (mL) | Solid state form |
| MeOH | 10.7 | 4 | Water | 10 | Crystalline base form |
| MeOH | 9.6 | 2 | Acetone | 6 | Too little solid to analyse* |
| MeOH | 10.7 | 2 | MTBE | 6 | Form A, Form B and channel |
| | | | | | solvate |
| MeOH | 10.8 | 2 | Toluene | 6 | No solid* |
| DMF | 25 | 2 | Water | 6 | Unidentified form |
| DMF | 25.6 | 2 | Acetone | 6 | Too little solid to analyse* |
| DMF | 25 | 2 | MTBE | 3 | Form C with a little Form A |
| DMF | 24.9 | 1 | Toluene | 3 | No solid* |
| Benzyl alcohol | 25 | 2 | Isopropyl | 6 | Form A and Form C |
| | | | acetate | | |
| Benzyl alcohol | 25.5 | 2 | Acetone | 6 | Unstable solvate* |
| Benzyl alcohol | 24.7 | 2 | MTBE | 6 | Forms 1, 2, 3 and an unknown |
| | | | | | form |
| Benzyl alcohol | 25.5 | 2 | Toluene | 6 | No solid* |
| Pyridine | 25.3 | 1 | Water | 3 | Too little solid to analyse |
| Pyridine | 25.5 | 1 | Acetone | 3 | Free base form* |
| Pyridine | 25.2 | 1 | MTBE | 3 | Form A and Form C |
| Pyridine | 25.4 | 1 | Toluene | 3 | Forms 1, 3 and an unknown |
| | | | | | form* |
| DMSO | 25.5 | 1 | Water | 3 | Crystalline base form |
| DMSO | 25.6 | 1 | Acetone | 3 | No solid* |
| DMSO | 24.6 | 1 | EtOAc | 3 | No solid** |
| DMSO | 24.8 | 1 | Toluene | 3 | No solid |

*sample put in freezer after 3 days; piece of metal wire added after one week.
**piece of metal wire added after three weeks.

### Reversed Anti-Solvent Crystallisation Experiments

Five solvents where the X842 HBr salt has a high solubility were used together with six antisolvents in which the solubility was low. Saturated solutions were prepared by adding solvent in portions of 100 μL until the solutions were clear. The solutions were placed in a cooling block at 18° C. Thereafter, they were filtered through 0.2 um syringe filters and returned to RT, see table 5. Finally, the solutions were added into the anti-solvent (solvent 2), see table 6. The samples were analysed after 3 to 5 days, unless indicated otherwise.

TABLE 5
Saturated solutions of X842 HBr salt

| Solvent 1 | Amount salt (mg) |
|---|---|
| MeOH | 50.5 |
| DMF | 149.4 |
| Benzyl alcohol | 49.7 |
| Pyridine | 150.5 |
| DMSO | 149.4 |

TABLE 6
Results of reversed anti-solvent experiments

| Saturated | Saturated | | | | |
|---|---|---|---|---|---|
| solvent 1 | solvent 1 (mL) | Solvent 2 | Solvent 2 (mL) | Solid state form | Comment |
| MeOH | 0.4 | Water | 7 | Crystalline base form | Precipitated |
| | | | | and possibly Form A | immediately |
| MeOH | 0.4 | Acetone | 7 | Forms A and C* | Precipitated after |
| | | | | | few days in freezer |
| MeOH | 0.4 | MTBE | 7 | Form B | Precipitated |
| | | | | | immediately |
| MeOH | 0.3 | Toluene | 7 | Form B | Precipitated after |
| | | | | | one hour |
| DMF | 0.05 | Water | 1 | Form B and | Precipitated |
| | | | | crystalline base form | immediately |
| DMF | 0.05 | Acetone | 1 | Form C | Precipitated after 3 |
| | | | | | days |
| DMF | 0.05 | MTBE | 1 | Form A | Precipitated |
| | | | | | immediately |
| DMF | 0.05 | Toluene | 1 | Forms A and B** | Precipitated |
| | | | | | immediately |
| Benzyl | 0.1 | Isopropyl | 4 | Form B | Precipitated |
| alcohol | | acetate | | | immediately |
| Benzyl | 0.1 | Acetone | 4 | Forms B and C* | A small piece of |
| alcohol | | | | | metal wire was also |
| | | | | | added |
| Benzyl | 0.1 | MTBE | 4 | Form B | Precipitated |
| alcohol | | | | | immediately |
| Benzyl | 0.1 | Toluene | 4 | Forms A and B** | Precipitated |
| alcohol | | | | | immediately |
| Pyridine | 0.05 | Water | 1 | Crystalline base | Precipitated |
| | | | | forms | immediately |
| Pyridine | 0.05 | Acetone | 1 | Form Cand | Precipitation after |
| | | | | crystalline base form | three days |
| Pyridine | 0.05 | MTBE | 1 | Form B | Precipitated |
| | | | | | immediately |
| Pyridine | 0.05 | Toluene | 1 | Form B with an | Precipitated |
| | | | | unknown peak** | immediately |
| DMSO | 0.05 | Water | 1 | Form B and | Precipitated |
| | | | | crystalline base form | immediately |
| DMSO | 0.05 | Acetone | 1 | DMSO solvate* | A small piece of |
| | | | | | metal wire was also |
| | | | | | added |
| DMSO | 0.05 | EtOAc | 1 | DMSO solvate | Precipitated |
| | | | | | immediately |
| DMSO | 0.05 | Toluene | 1 | DMSO solvate and | A small piece of |
| | | | | Form C* | metal wire was also |
| | | | | | added. Very little |
| | | | | | precipitation after |
| | | | | | 1.5 weeks |

*Put in freezer after three days; put in freezer after 1 week; analysed after 1.5 weeks.
**The mixture was slurried in RT for five-six days following analysis, after which Form C was formed.
*** The mixture was slurried in RT for six days following analysis, after which Form A was formed.

### Cooling Experiments

Cooling experiments were performed in five different solvents where the solubility of the drug substance is high enough to dissolve a reasonable amount. The solutions were then cooled to produce a solid phase. Saturated solutions were prepared at RT. The temperature was then increased to 40° C., thereafter the solutions were filtered through 0.2 m syringe filters, and then heated to 40° C. once again, after which they were placed in a refrigerator at 5° C. A piece of metal wire was inserted into the solutions after 5 days. The samples were analysed after 1.5 weeks The results are shown below in table 7.

TABLE 7
Results of the cooling experiments

| Amount | | Solvent | |
|---|---|---|---|
| salt (mg) | Solvent | volume (mL) | Solid state form |
| 39.5 | Methanol | 2 | Form B |
| 20.4 | Ethanol | 4.5 | Form A and Form C* |
| 49.8 | DMF | 0.2 | No solid* |
| 49.9 | Pyridine | 0.2 | No solid* |
| 50.8 | Benzyl alcohol | 0.6 | No solid |

*Placed in freezer after 1.5 weeks; analysed after 2 weeks.

The XRPD peaks for Form A, as obtained from the synthesis described in example 1, are listed in Table 8 below. The diffractogram for Form A is shown in FIG. 1.

TABLE 8
XRPD peaks for Form A

| Position | Height | FWHM Left | d-spacing | Rel. Int. * |
|---|---|---|---|---|
| [°2θ] | [cts] | [°2θ] | [Å] | [%] |
| 5.4128 | 7752.45 | 0.0936 | 16.31377 | 100.00 |
| 9.3573 | 466.73 | 0.0936 | 9.44376 | 6.02 |
| 11.7913 | 986.36 | 0.1092 | 7.49923 | 12.72 |
| 13.4558 | 1459.55 | 0.1404 | 6.57507 | 18.83 |
| 13.8044 | 320.87 | 0.1092 | 6.40981 | 4.14 |
| 14.1212 | 609.72 | 0.0780 | 6.26670 | 7.86 |
| 14.3519 | 1185.92 | 0.1248 | 6.16651 | 15.30 |
| 14.7501 | 980.40 | 0.0936 | 6.00092 | 12.65 |
| 14.8888 | 692.53 | 0.0780 | 5.94532 | 8.93 |
| 15.4246 | 1070.46 | 0.1248 | 5.73997 | 13.81 |
| 16.0343 | 497.76 | 0.1248 | 5.52308 | 6.42 |
| 17.3103 | 1143.98 | 0.1248 | 5.11872 | 14.76 |
| 18.2721 | 1513.68 | 0.1248 | 4.85138 | 19.53 |
| 18.8840 | 797.17 | 0.1248 | 4.69553 | 10.28 |
| 19.3552 | 2727.03 | 0.1092 | 4.58227 | 35.18 |
| 19.8931 | 1103.55 | 0.1092 | 4.45956 | 14.23 |
| 20.6294 | 1190.79 | 0.1248 | 4.30203 | 15.36 |
| 21.1933 | 712.90 | 0.1092 | 4.18882 | 9.20 |
| 21.4795 | 936.93 | 0.1248 | 4.13365 | 12.09 |
| 21.6736 | 506.16 | 0.1092 | 4.09708 | 6.53 |
| 21.8795 | 476.07 | 0.1092 | 4.05897 | 6.14 |
| 22.4475 | 4459.32 | 0.1248 | 3.95753 | 57.52 |
| 23.3557 | 872.49 | 0.1092 | 3.80567 | 11.25 |
| 23.8853 | 2701.85 | 0.0936 | 3.72247 | 34.85 |
| 24.0241 | 1788.18 | 0.0780 | 3.70127 | 23.07 |
| 24.2274 | 753.62 | 0.1248 | 3.67067 | 9.72 |
| 24.8577 | 473.03 | 0.1092 | 3.57901 | 6.10 |
| 25.1616 | 551.21 | 0.1092 | 3.53646 | 7.11 |
| 25.4485 | 3536.37 | 0.1248 | 3.49724 | 45.62 |
| 25.7560 | 1340.25 | 0.1092 | 3.45618 | 17.29 |
| 26.2793 | 1117.70 | 0.1248 | 3.38853 | 14.42 |
| 26.4710 | 1667.31 | 0.0936 | 3.36442 | 21.51 |
| 26.7546 | 1378.81 | 0.1404 | 3.32941 | 17.79 |
| 26.9694 | 443.21 | 0.0780 | 3.30337 | 5.72 |
| 27.4639 | 701.10 | 0.1092 | 3.24501 | 9.04 |
| 27.6273 | 466.33 | 0.0780 | 3.22619 | 6.02 |
| 28.0639 | 1181.95 | 0.1248 | 3.17698 | 15.25 |
| 28.4535 | 467.07 | 0.1092 | 3.13435 | 6.02 |
| 28.7055 | 589.29 | 0.1092 | 3.10741 | 7.60 |
| 29.0224 | 398.95 | 0.1248 | 3.07419 | 5.15 |
| 29.4948 | 623.34 | 0.1092 | 3.02602 | 8.04 |
| 29.9169 | 508.84 | 0.1248 | 2.98428 | 6.56 |
| 30.0661 | 342.23 | 0.0936 | 2.96982 | 4.41 |
| 31.2111 | 1941.88 | 0.1248 | 2.86342 | 25.05 |
| 32.2432 | 585.51 | 0.1248 | 2.77409 | 7.55 |
| 32.9478 | 1945.85 | 0.1092 | 2.71635 | 25.10 |
| 34.3716 | 528.81 | 0.1092 | 2.60702 | 6.82 |

* The relative intensity depends on the particle orientation, crystallite size/shape, strain and specimen thickness

The XRPD peaks for Form B, as obtained from a slurry in methanol/toluene 4:1 (“sample 1”) are listed in Table 9 below. The diffractogram for Form B, sample 1 is shown in FIG. 2. The diffractogram for Form B as obtained from a slurry in methanol/water 4:1 (“sample 2”) is shown in FIG. 3.

TABLE 9
XRPD peaks for Form B, sample 1

| Position | Height | FWHM Left | d-spacing | Rel. Int. * |
|---|---|---|---|---|
| [°2θ] | [cts] | [°2θ] | [Å] | [%] |
| 7.5976 | 1106.42 | 0.0780 | 11.62660 | 24.00 |
| 9.0580 | 1428.43 | 0.0936 | 9.75514 | 30.98 |
| 10.6519 | 622.79 | 0.0780 | 8.29869 | 13.51 |
| 11.3960 | 322.44 | 0.0936 | 7.75846 | 6.99 |
| 12.1591 | 1672.79 | 0.0936 | 7.27321 | 36.28 |
| 12.9693 | 184.47 | 0.0780 | 6.82058 | 4.00 |
| 14.5491 | 1676.67 | 0.0936 | 6.08334 | 36.36 |
| 16.1637 | 596.60 | 0.1092 | 5.47913 | 12.94 |
| 16.6510 | 355.70 | 0.1092 | 5.31988 | 7.71 |
| 17.9653 | 388.13 | 0.0936 | 4.93353 | 8.42 |
| 20.3743 | 1048.28 | 0.0780 | 4.35533 | 22.73 |
| 20.5204 | 625.51 | 0.0624 | 4.32465 | 13.57 |
| 20.6454 | 746.43 | 0.0780 | 4.29873 | 16.19 |
| 21.1440 | 1516.25 | 0.0780 | 4.19847 | 32.88 |
| 21.4160 | 1176.57 | 0.0780 | 4.14577 | 25.52 |
| 21.8358 | 1342.40 | 0.1092 | 4.06700 | 29.11 |
| 22.1910 | 500.36 | 0.0780 | 4.00270 | 10.85 |
| 22.5700 | 657.74 | 0.0936 | 3.93633 | 14.26 |
| 23.2946 | 309.49 | 0.0780 | 3.81550 | 6.71 |
| 23.6406 | 2035.32 | 0.0780 | 3.76045 | 44.14 |
| 23.8999 | 266.05 | 0.0780 | 3.72022 | 5.77 |
| 24.4232 | 501.34 | 0.0780 | 3.64169 | 10.87 |
| 24.7978 | 4610.89 | 0.1092 | 3.58752 | 100.00 |
| 25.4081 | 805.90 | 0.0780 | 3.50271 | 17.48 |
| 25.7731 | 2044.91 | 0.1092 | 3.45392 | 44.35 |
| 26.2568 | 571.06 | 0.0780 | 3.39138 | 12.38 |
| 26.5506 | 508.04 | 0.0624 | 3.35452 | 11.02 |
| 26.6564 | 407.64 | 0.0624 | 3.34145 | 8.84 |
| 27.0367 | 372.08 | 0.0936 | 3.29531 | 8.07 |
| 27.2532 | 1507.11 | 0.0936 | 3.26961 | 32.69 |
| 27.8072 | 837.46 | 0.0936 | 3.20572 | 18.16 |
| 29.2064 | 389.57 | 0.0936 | 3.05524 | 8.45 |
| 29.6393 | 1002.41 | 0.1092 | 3.01160 | 21.74 |
| 29.9312 | 479.71 | 0.1092 | 2.98289 | 10.40 |
| 30.5110 | 306.10 | 0.1092 | 2.92752 | 6.64 |
| 30.9337 | 889.35 | 0.0624 | 2.88847 | 19.29 |
| 31.4839 | 282.29 | 0.1560 | 2.83923 | 6.12 |
| 32.0474 | 439.28 | 0.1248 | 2.79059 | 9.53 |

* The relative intensity depends on the particle orientation, crystallite size/shape, strain and specimen thickness

The XRPD peaks for Form C, as obtained from a slurry in DMF/toluene 1:1, are listed in Table 10 below. The diffractogram for Form C is shown in FIG. 4.

TABLE 10
XRPD peaks for Form C

| Position | Height | FWHM Left | d-spacing | Rel. Int. * |
|---|---|---|---|---|
| [°2θ] | [cts] | [°2θ] | [Å] | [%] |
| 8.5128 | 353.51 | 0.0780 | 10.37867 | 5.18 |
| 9.1128 | 1035.86 | 0.0780 | 9.69653 | 15.19 |
| 10.3633 | 1026.34 | 0.0936 | 8.52914 | 15.05 |
| 11.7023 | 1144.35 | 0.1404 | 7.55605 | 16.78 |
| 12.6223 | 636.66 | 0.0624 | 7.00732 | 9.34 |
| 13.0990 | 1045.03 | 0.0936 | 6.75338 | 15.33 |
| 15.5579 | 471.22 | 0.0780 | 5.69110 | 6.91 |
| 16.1149 | 464.02 | 0.0780 | 5.49562 | 6.81 |
| 16.8726 | 487.53 | 0.0936 | 5.25048 | 7.15 |
| 18.1699 | 925.84 | 0.1248 | 4.87845 | 13.58 |
| 18.5584 | 2988.22 | 0.0936 | 4.77718 | 43.83 |
| 18.8785 | 690.46 | 0.0936 | 4.69690 | 10.13 |
| 19.3351 | 254.72 | 0.0780 | 4.58700 | 3.74 |
| 19.9382 | 6818.49 | 0.1092 | 4.44959 | 100.00 |
| 20.4802 | 571.35 | 0.0780 | 4.33304 | 8.38 |
| 22.0690 | 1309.92 | 0.0780 | 4.02455 | 19.21 |
| 22.4389 | 2050.78 | 0.1092 | 3.95903 | 30.08 |
| 22.7928 | 1573.86 | 0.0936 | 3.89835 | 23.08 |
| 23.0626 | 1345.59 | 0.1248 | 3.85336 | 19.73 |
| 23.4185 | 5533.78 | 0.0936 | 3.79560 | 81.16 |
| 24.2728 | 5199.85 | 0.1092 | 3.66391 | 76.26 |
| 24.5124 | 5206.10 | 0.1092 | 3.62863 | 76.35 |
| 24.8108 | 1329.89 | 0.1404 | 3.58566 | 19.50 |
| 25.0322 | 409.12 | 0.0936 | 3.55444 | 6.00 |
| 25.2918 | 1325.41 | 0.0936 | 3.51855 | 19.44 |
| 25.5019 | 1680.17 | 0.1092 | 3.49004 | 24.64 |
| 25.9503 | 1026.49 | 0.0936 | 3.43074 | 15.05 |
| 26.5640 | 5689.90 | 0.0936 | 3.35286 | 83.45 |
| 26.8500 | 1549.51 | 0.0936 | 3.31779 | 22.73 |
| 27.7332 | 1546.45 | 0.0936 | 3.21410 | 22.68 |
| 28.1038 | 646.26 | 0.1092 | 3.17255 | 9.48 |
| 28.6264 | 389.46 | 0.0780 | 3.11581 | 5.71 |
| 28.9528 | 425.65 | 0.1092 | 3.08143 | 6.24 |
| 29.6636 | 368.30 | 0.0936 | 3.00919 | 5.40 |
| 29.8857 | 689.14 | 0.1248 | 2.98733 | 10.11 |
| 30.1515 | 470.43 | 0.1092 | 2.96160 | 6.90 |
| 31.1336 | 514.20 | 0.0936 | 2.87038 | 7.54 |
| 31.3253 | 474.04 | 0.0780 | 2.85325 | 6.95 |
| 31.7054 | 823.19 | 0.0936 | 2.81990 | 12.07 |
| 32.5752 | 1281.03 | 0.0936 | 2.74656 | 18.79 |
| 32.7484 | 530.39 | 0.0936 | 2.73243 | 7.78 |
| 34.1979 | 427.56 | 0.0936 | 2.61986 | 6.27 |
| 34.8306 | 2492.09 | 0.0936 | 2.57371 | 36.55 |

* The relative intensity depends on the particle orientation, crystallite size/shape, strain and specimen thickness

The different solvates were not deemed pharmaceutically viable and are therefore not described further in this disclosure.

### Example 3

### Thermogravimetric Analysis

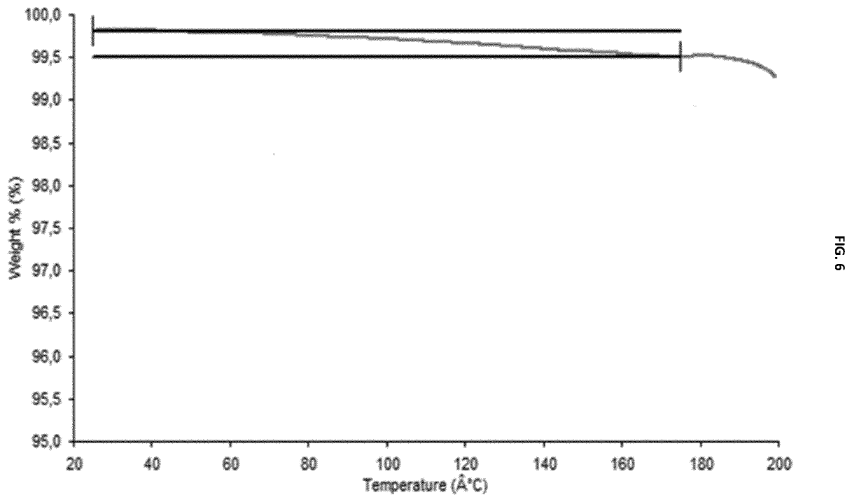

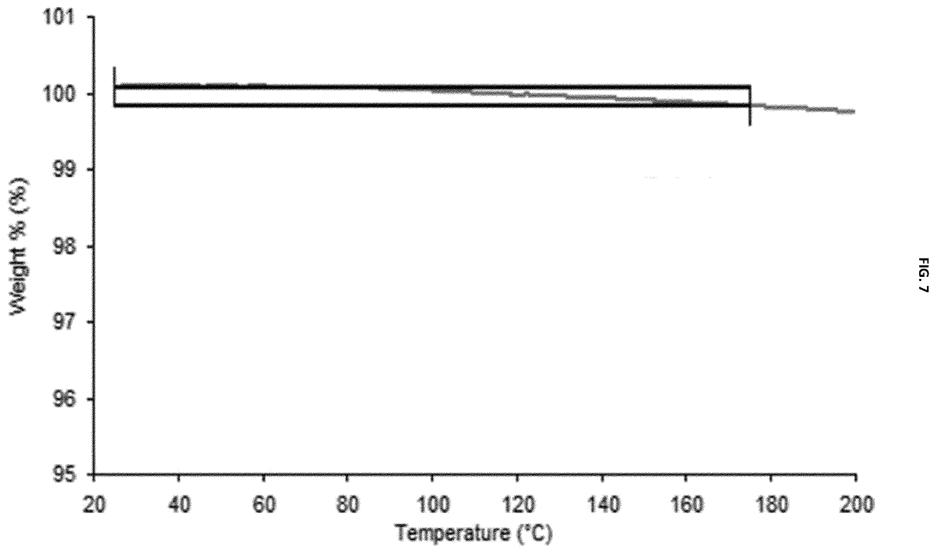

A sample of Form A, obtained from the synthesis described in Example 1, showed a weight loss of 0.3% upon heating up to 175° C. This confirms that Form A is an anhydrate. The TGA weight loss curve for Form A is shown in FIG. 5.

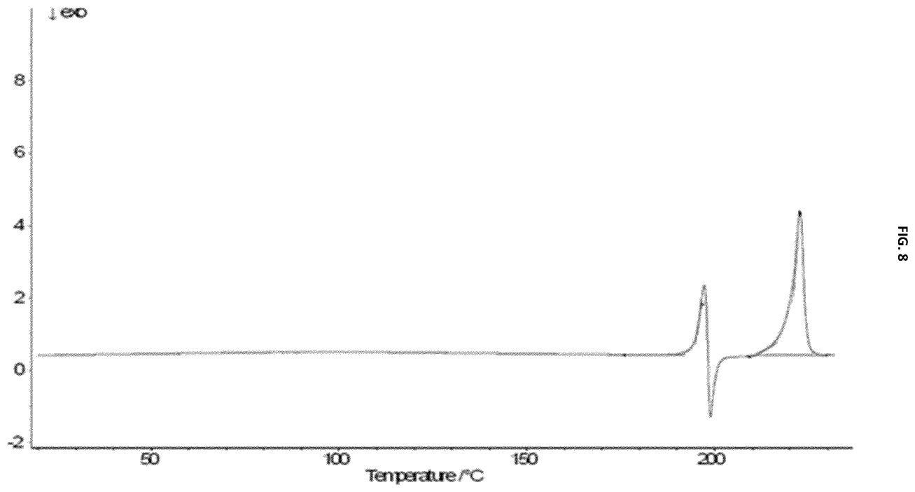

A sample of Form B (obtained from a slurry in methanol/toluene 4:1) showed a weight loss of 0.3% upon heating up to 175° C. This confirms that Form B also is an anhydrate. The TGA weight loss curve for Form A is shown in FIG. 6.

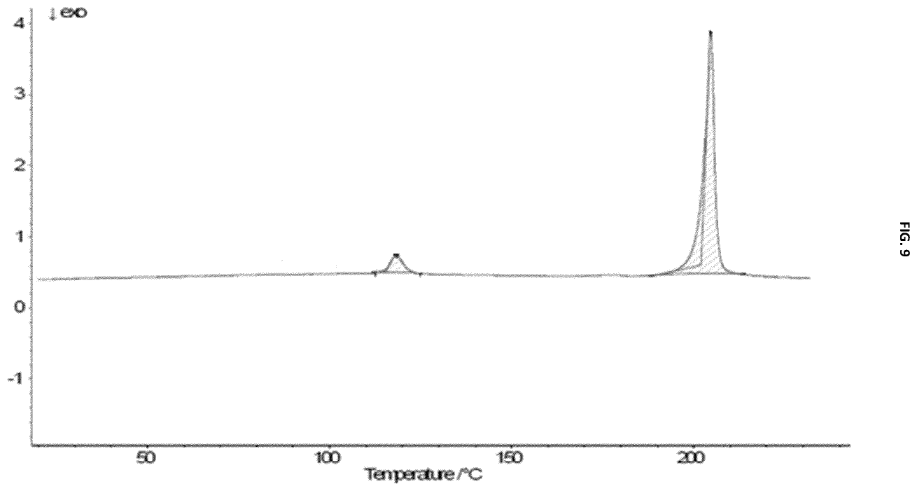

A sample of Form C (obtained from a slurry in DMF/toluene 1:1) showed a weight loss of 0.2% upon heating up to 175° C. This confirms that Form C is an anhydrate as well. The TGA weight loss curve for Form A is shown in FIG. 7.

### Example 4

### Differential Scanning Calorimetry (DSC) Analysis

A sample of Form A (obtained from the synthesis described in Example 1) displayed an endothermic event with an onset of about 195° C., directly followed (overlaid) by an exothermic event, interpreted as a crystallisation into another form. The new, unknown, form thereafter melted in a second endothermic event with an onset of about 220° C. The DSC thermogram of Form A is shown in FIG. 8.

A sample of Form B (obtained from a slurry in methanol/water 4:1) showed two endothermic events. First a small endothermic event was observed at about 118° C. (onset:about 116° C.), which is interpreted as a solid-state transition into another form. The new (unknown) form then started melting at about 205° C. (onset:about 202° C.). The DSC thermogram of Form B is shown in FIG. 9.

A sample of Form C (as obtained from a slurry in ethanol) showed one thermal event at about 223° C. with an onset temperature of about 214° C. The DSC thermogram is shown in FIG. 10.

### Example 5

### Dynamic Vapour Sorption (DVS) Analysis

The hygroscopicity of Form A (as obtained from the synthesis described in Example 1), Form B (as obtained from a slurry in methanol/water 4:1) and Form C (as obtained from a slurry in ethanol) were investigated using GVS at 25° C. The weight change plot and the sorption isotherm plot for Form A showed a water uptake of ca 0.22% in the humidity range of 0-90%; see FIGS. 11A and 11B, respectively. Form A can thus be classified as non-hygroscopic.

For Form B, the weight change plot and the sorption isotherm plot showed a significant water uptake with increasing humidity. With a weight gain of about 3% at elevated humidities, it appears that a hydrate form is formed. Upon drying, Form B returned to the anhydrate; see FIGS. 12A and 12B, respectively.

For Form C, the weight change plot and the sorption isotherm plot showed a water uptake of only about 0.05% in the humidity range of 0-90%; see FIGS. 13A and 13B, respectively. Form C is thus classified as non-hygroscopic.

1. A crystalline HBr salt of linaprazan glurate.

2. The crystalline HBr salt of linaprazan glurate according to claim 1, wherein the crystalline HBr salt is stable at a relative humidity of 94% at room temperature.

3. The crystalline HBr salt of linaprazan glurate according to claim 1, which is an anhydrate.

4. The crystalline HBr salt of linaprazan glurate according to claim 1, which is Form A, having an XRPD pattern, obtained with CuKα1-radiation, with at least two peaks at °2θ values selected from the list consisting of 5.4±0.2, 18.3±0.2, 19.4±0.2, 22.4±0.2, 23.9±0.2, 24.0±0.2, 25.4±0.2, 26.5±0.2, 31.2±0.2, and 32.9±0.2.

5. The crystalline HBr salt of linaprazan glurate according to claim 1, which is Form A, having an XRPD pattern, obtained with CuKα-radiation, with at least peaks at °2θ values of 5.4±0.2, 19.4±0.2, 22.4±0.2 and 25.4±0.2.

6. (canceled)

7. The crystalline HBr salt of linaprazan glurate according to claim 1, which is Form A, having an XRPD pattern, obtained with CuKα-radiation, substantially as shown in FIG. 1.

8. The crystalline HBr salt of linaprazan glurate according to claim 1, which is Form A, having a DSC curve comprising an endotherm between about 194° C. and about 198° C., such as at about 196° C.

9. (canceled)

10. The crystalline HBr salt of linaprazan glurate according to claim 1, which is Form B, having an XRPD pattern, obtained with CuKα1-radiation, with at least two peaks at °2θ values selected from the list consisting of 9.1±0.2, 12.2±0.2, 14.5±0.2, 21.1±0.2, 21.4±0.2, 21.8±0.2, 23.6±0.2, 24.8±0.2, 25.8±0.2, and 27.3±0.2.

11. The crystalline HBr salt of linaprazan glurate according to claim 10, wherein Form B has an XRPD pattern, obtained with CuKα1-radiation, with at least peaks at 020 values 14.5±0.2, 23.6±0.2, 24.8±0.2 and 25.8±0.2.

12. (canceled)

13. The crystalline HBr salt of linaprazan glurate according to claim 10, wherein Form B has an XRPD pattern, obtained with CuKα-radiation, substantially as shown in FIG. 2 or FIG. 3.

14. The crystalline HBr salt of linaprazan glurate according to claim 10, wherein Form B has a DSC curve comprising an endotherm between about 115° C. and about 121° C., such as at about 118° C.

15. (canceled)

16. The crystalline HBr salt of linaprazan glurate according to claim 1, which is Form C, having an XRPD pattern, obtained with CuKα1-radiation, with at least two peaks at ° 20 values selected from the list consisting of 18.6±0.2, 19.9±0.2, 22.4±0.2, 22.8±0.2, 23.4±0.2, 24.3±0.2, 24.5±0.2, 25.5±0.2, 26.6±0.2, and 34.8±0.2.

17. The crystalline HBr salt of linaprazan glurate according to claim 16, wherein Form C has an XRPD pattern, obtained with CuKα1-radiation, with at least peaks at 020 values of 19.9±0.2, 23.4±0.2, 24.5±0.2 and 26.6±0.2.

18. (canceled)

19. The crystalline HBr salt of linaprazan glurate according to claim 16, wherein Form C has an XRPD pattern, obtained with CuKα-radiation, substantially as shown in FIG. 4.

20. The crystalline HBr salt of linaprazan glurate according to claim 16, wherein Form C has a DSC curve comprising an endotherm between about 214° C. and about 230° C., such as at about 226° C.

21. The crystalline HBr salt of linaprazan glurate according to claim 1, having a crystallinity of greater than 99%.

22. A pharmaceutical composition comprising a therapeutically effective amount of a crystalline HBr salt of linaprazan glurate according to claim 1, in association with one or more pharmaceutically acceptable excipients.

23. (canceled)

24. A method of treating or preventing a gastrointestinal inflammatory disease or a gastric acid related disease, comprising administering the crystalline HBr salt of linaprazan glurate according to claim 1.

25. The method according to claim 24, wherein the gastrointestinal inflammatory disease or the gastric acid related disease is gastritis, gastroesophageal reflux disease (GERD), erosive gastroesophageal reflux disease (eGERD), H. pylori infection, Zollinger-Ellison syndrome, peptic ulcer disease (including gastric ulcers and duodenal ulcers), bleeding gastric ulcer, symptoms of gastroesophageal reflux disease (including heartburn, regurgitation and nausea), gastrinoma or acute upper gastrointestinal bleeding.

26. The method according to claim 24, wherein the gastrointestinal inflammatory disease or the gastric acid related disease is erosive gastroesophageal reflux disease (eGERD).
